(19) United States
(12) Patent Application Publication
(10) Pub. No.: US 2025/0380965 A1
(43) Pub. Date: Dec. 18, 2025

(54) MACHINE VISION BASED ELECTRODE IMPLANTATION METHOD AND SYSTEM

(71) Applicant: SHANGHAI STAIRMED TECHNOLOGY CO., LTD., Shanghai (CN)

(72) Inventors: Zhengtuo ZHAO, Shanghai (CN); Xue LI, Shanghai (CN); Ruolan ZHAO, Shanghai (CN)

(21) Appl. No.: 18/877,207

(22) PCT Filed: Jun. 29, 2022
(86) PCT No.: PCT/CN2022/102359

§ 371 Date: Dec. 19, 2024

(30) Foreign Application Priority Data
Jun. 20, 2022 (CN) 202210696347.9

(51) Int. Cl. A61B 17/34; A61B 90/00; G01B 11/00; G06T 7/00; G06T 7/168; G06T 7/80

(52) U.S. Cl. CPC A61B 17/3468; A61B 90/37; G01B 11/002; G06T 7/0012; G06T 7/168; G06T 7/85; A61B 2090/371; G06T 2207/10021; G06T 2207/20182; G06T 2207/30016; G06T 2207/30101

(57) ABSTRACT

The present disclosure relates to a machine vision based electrode implantation method and system. The method includes: performing arithmetic processing on a first image captured by a first camera and a second image captured by a second camera for a brain surface, wherein, a vascular area mask of the brain surface is obtained to determine an implantable area in a brain surface image; selecting at least one implantation position in the implantable area, so as to determine an implantation sequence of the electrodes; matching the imaging of the first camera and the second camera to obtain a transformation matrix, and determining an intersection point based on the imaging of the first camera and the second camera as a predicted landing point of the an implantation apparatus.

### TECHNICAL FIELD

The present disclosure relates to the field of life science technology, and in particular to a machine vision based electrode implantation method and system.

### BACKGROUND

In the field of a neurosurgery robot, the implantation of a flexible electrode to a brain surface is involved. During the implantation process, the implantation apparatus first passes through the electrode, and then drive the electrode that is to be implanted onto the brain surface. However, the brain surface operation area is mostly a millimeter-level small window, and by depending on a robotic arm or an external stepper motor to initially move to above the small window, the implantable site of the blood vessel is analyzed and avoided when controlling the implantation of the implantation tool. In order to lessen or reduce the bleeding when the electrode is implanted, it is necessary to automatically recognize the implantable area. Because of the complex distribution of the blood vessel on the brain surface of an animal, there are many capillaries; and the image quality is very strict with the imaging condition, and the change of the illumination may greatly affect the recognition effect of the blood vessel. Therefore, there is a need for a stable optical system to ensure the image quality, as well as a stable algorithm that can recognize a cerebral vessel and provide an implantation area.

Further, during the electrode implantation, it is required to recognize a correspondence relationship between the implantation brain area and the electrode channel, and it is necessary to number the implantation position. During the electrode implantation process, there are often a plurality of sites to be implanted. However, the electrode has a limited length, and it is necessary to consider that the electrode is not pulled and its arrangement relationship with other electrodes, which raises the requirement for the implantation position sequence of the electrode. In addition, since the implantation tool is not necessarily completely vertical, and the brain surface fluctuates, it is required to perform accurate implantation so as to strictly control the implantation angle and position between the implantation tool and the surface, and accurately judge a spatial position of the implantation tool. Therefore, it is also necessary to design a corresponding stereoscopic microscopic imaging system to monitor a position of the implantation tool in real time and predict its landing point on the brain surface.

### SUMMARY

The present application provides a machine vision based electrode implantation method and system.

According to a first aspect of the embodiment of the present disclosure, a machine vision based electrode implantation method is provided. The method includes: capturing a first image by a first camera for a brain surface, and capturing a second image by a second camera for the brain surface; performing arithmetic processing on the first image and the second image, wherein, a vascular area mask of the brain surface is obtained based on a vascular segmentation algorithm to determine an implantable area in a brain surface image; selecting at least one implantation position in the implantable area, and calculating a distance between the at least one implantation position and an electrode position according to a known electrode position, so as to determine an implantation sequence of the electrodes; matching imaging of the first camera and the second camera to obtain a transformation matrix, projecting a first straight line where the implantation position is situated in the imaging of the first camera onto the imaging of the second camera, and determining an intersection point between the first straight line and a second straight line where the implantation position is situated in the imaging of the second camera as a predicted landing point of an implantation apparatus; and controlling the implantation apparatus in real time according to the predicted landing point until an implantation point coincides with the predicted landing point.

According to a second aspect of the embodiment of the present disclosure, a machine vision based electrode implantation system is provided. The system includes: a first camera configured to capture a first image for a brain surface; a second camera configured to capture a second image for a brain surface; a vascular segmentation arithmetic unit configured to perform arithmetic processing on the first image and the second image, wherein a vascular area mask of the brain surface is obtained based on a vascular segmentation algorithm to determine an implantable area in a brain surface image; an implantation sequence determining unit configured to select at least one implantation position in the implantable area and calculate a distance between the at least one implantation position and an electrode position according to a known electrode position, so as to determine an implantation sequence of the electrodes; an implantation landing point prediction unit configured to match imaging of the first camera and the second camera to obtain a transformation matrix, project a first straight line where the implantation position is situated in the imaging of the first camera onto the imaging of the second camera, and determine an intersection point between the first straight line and a second straight line where the implantation position is situated in the imaging of the second camera as a predicted landing point of an implantation apparatus; and an implantation apparatus control unit configured to control the implantation apparatus in real time according to the predicted landing point until an implantation point coincides with the predicted landing point.

The embodiment according to the present disclosure has the advantages that it is suitable for a plurality of different imaging modes, in which the algorithm used has a favorable universality, and it is possible to achieve a favorable boundary segmentation and obtain a stable imaging recognition result.

Another advantage of the embodiment according to the present disclosure is that it is possible to provide an automatic vascular segmentation algorithm and reduce the calculation amount of the segmentation algorithm when the algorithm parameters are easily adjusted.

It should be appreciated that, the above-described advantages are not required to be implemented by all gathering in one or some specific embodiments, but may be partially scattered in different embodiments according to the present disclosure. The embodiments according to the present disclosure may have one or some of the above-described advantages, and may also alternatively or additionally have other advantages.

Other features of the present invention and advantages thereof will become more explicit by way of the following detailed description of the exemplary embodiments of the present invention with reference to the accompanying drawings.

### BRIEF DESCRIPTION OF THE DRAWINGS

FIG. 1 is a schematic view showing a machine vision based electrode implantation system according to embodiments of the present disclosure.

FIG. 2 is a configuration view showing a machine vision based electrode implantation system according to embodiments of the present disclosure.

FIG. 3 is a flowchart showing a vascular segmentation algorithm according to embodiments of the present disclosure.

FIG. 4 is a schematic view showing steps of a vascular segmentation algorithm according to embodiments of the present disclosure.

FIG. 5 is an effect view showing a vascular segmentation algorithm according to embodiments of the present disclosure.

FIG. 6 is a schematic view showing implantable position selection and path planning of a brain surface electrode according to embodiments of the present disclosure.

FIG. 7 is a schematic view showing landing point prediction of an implantation tool according to embodiments of the present disclosure.

FIG. 8 is a flowchart showing an implantation tool control algorithm according to embodiments of the present disclosure.

FIG. 9 is a schematic view showing steps of a machine vision based electrode implantation method according to embodiments of the present disclosure.

### DETAILED DESCRIPTION

Various exemplary embodiments of the present disclosure will now be described below in detail with reference to the accompanying drawings. It should be noted that: the relative arrangements, numerical expressions and numerical values of the members and steps elaborated in these embodiments do not limit the scope of the present disclosure, unless specifically stated otherwise.

The following descriptions of at least one exemplary embodiment which are in fact merely illustrative, shall by no means serve as any delimitation on the present disclosure as well as its application or use. In other words, the structures and methods herein are shown in an exemplary manner to illustrate different embodiments of the structures and methods in the present disclosure. However, those skilled in the art will understand that they only describe exemplary methods of the present application that may be practiced, but not in an exhaustive way. Furthermore, the accompanying drawings are not necessarily drawn to scale, and some features might be exaggerated to show details of specific assemblies.

The techniques, methods, and devices known to those of ordinary skill in the relevant art might not be discussed in detail. However, the techniques, methods, and devices shall be considered as part of the granted description where appropriate.

During the electrode implantation process of the brain surface, it is necessary to perform blood vessel recognition, implantation site selection, implantation path planning and implantation tool positioning.

Generally, the blood vessel recognition on the brain surface mainly includes area segmentation for brain surface imaging, that is, distinguishing a vascular area from a non-vascular area. The area segmentation implementation methods at least include threshold partition, edge detection, segmentation methods based on mathematical morphology and the like.

Specifically, threshold partition is the most common segmentation method of parallel direct detection areas, and also the most simple segmentation method at the same time. This method generally has a certain hypothesis on the image. Suppose that the target and the background of the image occupy different gray-scale ranges, and the gray-scale value difference between adjacent pixels inside the target and background is small, but the gray-scale value difference between the pixels at the interface of the target and background is large. If an appropriate gray-scale threshold T is selected, and then the gray-scale value of each pixel in the image is compared with the threshold T, the pixels may be divided into two categories according to a comparison result: the pixels with a gray-scale value greater than a threshold are in one category and assigned a value of 1; the pixels with a gray-scale value less than the threshold are in another category and assigned a value of 0, so that a binary image is obtained and the target is extracted from the background. Normally, a segmentation threshold is determined according to prior knowledge, and it may also be determined by gray-scale histogram characteristics and statistical decision methods. The gray-scale histogram of the image may present a double peak and one valley shape. The two peaks correspond to the central gray-scale of the target and the central gray-scale of the background respectively, and the boundary point is located around the target, and its gray-scale is between the target gray-scale and the background gray-scale, so that the gray-scale of the boundary corresponds to the valley point between the two peaks. In order to minimize the pixel misclassification probability, the gray-scale of the valley point serves as a segmentation threshold. Because of the irregularity of histogram, it is difficult to determine a valley value of histogram, so that it is necessary to design a specific method to make a search. At present, there are many methods which may determine an optimal threshold (valley bottom), such as a method of calculating Gaussian model parameters and a method of fitting a histogram curve for evaluation of an extreme value.

The segmentation method based on a threshold has the advantages of simple calculation and high operation efficiency. However, this method is sensitive to noise and gray-scale diversity without considering the spatial characteristics, and it is difficult to obtain an accurate segmentation threshold for images with slight gray-scale difference between target and background gray-scales. In practical application, a satisfactory effect may be obtained in collaborative use with other image segmentation methods. In the prior art, when blood vessels are extracted from ToF (Time-of-flight) magnetic resonance angiography images, a statistical model based on a physical model of blood flow is provided. In order to improve the segmentation capability of the blood vessel, the velocity and phase information of PCA are fused, and two different statistical models are segmented by using an adaptive local threshold method and a single global threshold method respectively, so that the aneurysm with a very low signal in the vicinity thereof may achieve a favorable segmentation effect. In the prior art, a local threshold and a global threshold are also combined to perform three-dimensional reconstruction on the cerebrovascular image, so that the contrast of small blood vessels may be enhanced by using a local threshold, and the target blood vessels may be extracted from the background by using a global threshold.

Edge detection is a parallel boundary segmentation technology based on gray-scale discontinuity, and a first step of all boundary-based segmentation methods. Since the edge is a boundary line between the target and the background, the target and the background may be distinguished by extracting the edge. Edge detection is generally realized by using a difference between the target and the background in a particular characteristic, such as gray-scale, color, and texture. Edge detection is generally often accomplished by a first or second derivative, but in an actual digital image, deviation is to use difference operation approximation instead of differential operation. Since the points in the image on both sides of the edge have abrupt gray-scale values, these points will have a large differential value, and when the direction of differentiation is perpendicular to the boundary, the differential value is maximum. It may be seen that, differentiation is a directional operation, which is used to measure a gray-scale level in the direction of differentiation.

The basic principle of the segmentation method based on mathematical morphology is to perform basic operations on the image by using structural elements with certain morphology to in order to achieve the purpose of image analysis and recognition. The basic operations of mathematical morphology include dilation and erosion, as well as opening and closing operations formed by a combination thereof. The opening operation is corrosion followed expansion, and the closing operation is expansion followed by corrosion. All operations have respective characteristics in image processing. Dilation enlarges the image and corrosion narrows the image. The opening operation and the closing operation may both make the contour of the image become smooth, but the two operations have opposite effects. The opening operation may break the narrow discontinuity and eliminate the slim protrusions. The closing operation may eliminate small pores in the image, fill the cracks in the contour line, and merge narrow gaps and slim barriers. Combined with the specific features of the image, different mathematical morphology algorithms may be deduced and combined according to these basic operations, for processing and analyzing the shape and structure of the image, such as edge detection, image filtering, feature extraction and image enhancement. The processing algorithms of medical images commonly used are top-hat transformation and watershed transformation.

In order to implement the above-described different methods, neural network is also applied to image area segmentation in practice. On the one hand, neural network may perform learning. On the other hand, during the training process, boundary segmentation may be performed by using the nonlinearity of the network. Its shortcoming is that every time when a new feature is added to the network system, it is necessary to perform learning and training again, and its debugging process is also very complicated. In order to allow the network system to classify boundaries in the feature by using its learnability, it is necessary to select as many features of an object as possible. The algorithm widely applied during a learning process is post-propagation algorithm. Since the training data set determines the learning, the magnitude of the training data amount determines the learning process.

Further, the positioning of the implantation tool mainly includes hand-eye calibration in robot vision applications. Its aim is to obtain a relationship between the robot coordinate system and the camera coordinate system, and finally transfer a visual recognition result to the robot coordinate system.

There are two forms of hand-eye calibration in the art. According to different places where the camera is fixed, if the camera and the extremity of the robot are fixed together, it is referred to as “eye in hand”. If the camera is fixed on the base outside the robot, it is referred to as “eye to hand”.

In industry, the common hand-eye calibration methods are mainly divided into a nine-point calibration method and a calibration plate calibration method. Nine-point calibration directly establishes a coordinate transformation relationship between the camera and the manipulator. The indicator pointer at an extremity of the manipulator is made to be in contact with these nine points to obtain the coordinates in the robot coordinate system, and at the same time, the nine points in the initial screen are also identified by using a camera to obtain the pixel coordinates, so as to obtain nine groups of corresponding coordinates, and then solve a transformation matrix to obtain a transformation affine matrix between the image and the manipulator coordinates. The calibration plate calibration method uses a cross-hatch calibration plate or a circular grid calibration plate to obtain the internal and extrinsic parameters of the camera, so as to obtain a coordinate transformation relationship between the image and the manipulator.

The calibration of the monocular system is only applicable to the case where an observed object is in a horizontal plane, so that the depth information cannot be obtained. The conventional hand-eye calibration method may not accurately predict a landing point of the implantation tool on the brain surface. In contrast, the binocular system is divided into two categories according to a position relationship of the optical axis, that is, the binocular system with substantially parallel optical axes is a parallel binocular system and the binocular system with intersecting optical axes is a convergent binocular systems. Since there is a small overlapping range of visual field when the optical axes are parallel, the parallel binocular system is rarely used in the case of a small visual field, but generally used in the case where the working distance is much greater than the distance between lenses. However, if it is necessary to observe an object of a millimeter level, the convergent binocular system conforms more with the requirements.

However, there are still many shortcomings in the prior art. On the one hand, for the algorithm of image area segmentation, some algorithms provided for specific imaging modes are not universal, that is, they cannot be applied to other imaging modes. The boundary judgment of the blood vessel is performed based on the gray-scale gradient field of the pixel, but in an area with a low blood flow speed and a complex blood flow, the gradient value is often not high enough, which may result in reduced accuracy of the boundary judgment.

In the algorithm, suppose that the gray-scale distribution of each tissue is Gaussian distribution, but it is not so at all in actual cases, which results in deviation between the provided model and the clinical data. At the same time, a plurality of parameters involved in the image segmentation algorithm are required to be adjusted, and the parameter estimation process is very difficult. For some interactive algorithms, it is necessary to manually select a seed point or a termination point in the blood vessel, which affects the automation degree. In addition, on the whole, the segmentation method has a large calculation amount so that an expensive cost is involved in calculation.

On the other hand, for the positioning of the implantation tool, in order to accurately predict a landing point of the implantation tool on the brain surface in hand-eye calibration, the required imaging system has to use at least two cameras, that is, a binocular system is used. Moreover, because of the imaging characteristics of different binocular systems, it is necessary to use a convergent binocular system. In order to obtain high-definition imaging, it is necessary to also use a lens with a magnification of ×1 or ×2. In the case of the same camera pixel, the visual field has a small range and the lens has a very limited depth of field, generally about 1 mm. In this way, it is necessary to adjust a position of the camera to obtain high-definition imaging, which results in that the two cameras are not fixed relative to each other. However, the calibration of the binocular camera is only established when the camera position is relatively fixed, which means that the conventional binocular calibration method may not be directly applied to the brain surface electrode implantation system disclosed in the present application.

In order to solve the above-described technical problem, the inventors of the present application provide an improved machine vision based electrode implantation method and system, and in particular, relates to a brain surface electrode implantation method and system based on vascular segmentation processing on machine imaging. Generally, the technical solution of the present disclosure mainly includes performing automatic detection and avoiding a blood vessel during the electrode implantation process, and automatically detecting a plurality of candidates according to the sizes, numbers and shapes of the implantation tool and the implantation electrode. Brain partitions are projected on the brain surface by using the image registration and fusion technology, which facilitates positioning the brain partitions where the electrode is implanted during the operation process. After point selection is realized, the implantation system automatically moves to a selected position, and the electrode implantation apparatus is controlled to move accurately in a three-dimensional space based on the selected position and the angle of electrode implantation, and the accurate distance between the implantation apparatus and the brain surface is monitored in real time. The depth of electrode implantation is predicted according to the distance from the brain surface. In this process, the accurate movement and implantation of the electrode to the selected implantation site is realized by using technologies such as target tracking.

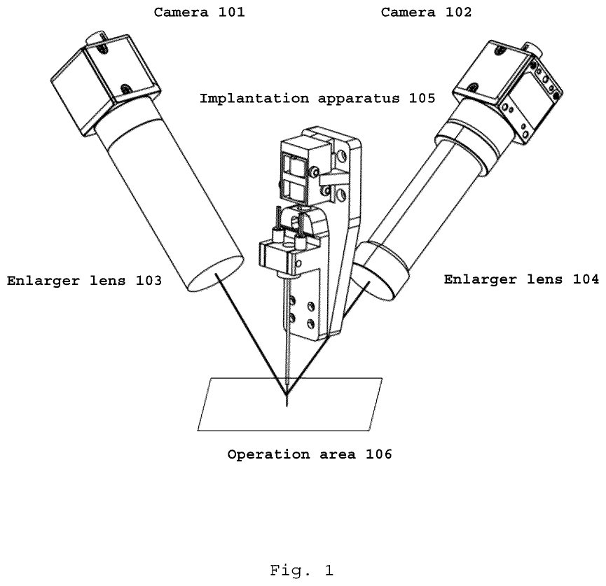

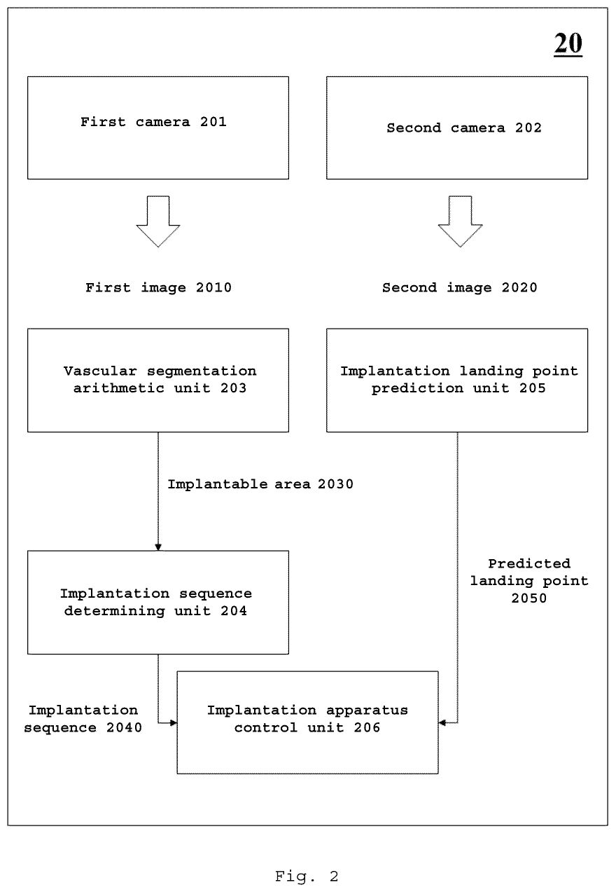

Hereinafter, the embodiment according to the present disclosure will be described in detail in conjunction with the accompanying drawings. First of all, FIGS. 1 and 2 show a schematic view and a configuration view of a machine vision based electrode implantation m according to embodiments of the present disclosure respectively. As shown in FIG. 1, the hardware structure of the brain surface electrode implantation system of the present application includes an optical system and a motion control system. Wherein, the optical system is mainly associated with two cameras 101 and 102 (hereinafter also referred to as “first camera” and “second camera”), such as a high-definition array CMOS industrial camera, which include telecentric lenses 103 and 104 for imaging enlarging of the operation area 106. The cameras 101 and 102 which have the same imaging plane, form a certain angle with each other on a projection plane, and are fixed on a rigid base plate, and a coaxial light source (not shown) is installed to shorten the exposure time and increase the frame rate. The light source may be an external point light source, which is mainly used to allow a uniform front light of the operation area 106 and avoid imaging blurring or overexposure of the implantation apparatus 105 and the operation area 106 and favorable for subsequent image and data processing. The light source may be white light or other light with a given wavelength. Preferably, due to the color influence of the brain surface and the blood vessel themselves, green light (for example, light with a wavelength of 495 nm to 570 nm) may be used to achieve better imaging effect.

In addition, three-axis slide tables are provided behind the cameras 101 and 102 respectively, which could adjust the enlarger lens 103 and 104 to reach a working distance, so as to image the position and angle of the implantation apparatus 105 relative to the operation area 106 in a plurality of orientations. FIG. 1 shows a non-limiting embodiment of the system disclosed in the present application, wherein the camera 101 and the camera 102 form an angle of about 90° degrees with each other in horizontal projection.

The motion control system which is mainly composed of three stepper motors, is configured to control the motion of the implantation apparatus 105 in three directions (±x, ±y and ±Z directions as shown in FIG. 1) of a certain spatial coordinate system. The cameras 101 and 102 are coupled to the motion control system respectively. In the non-limiting embodiment shown in FIG. 1, the three motors include one stepper motor and two micro-stepper motors (not shown) for controlling the motion in a ±z direction, for example, a stroke of 5 mm, for controlling the motion of the implantation apparatus 105 in the ±x and ±y directions respectively, that is, the movement of the area substantially parallel to the operation area 106.

Alternatively, the motion control system may also include a robotic arm with a similar motion control function. At this time, the camera 101/102 is disposed above the robotic arm. However, since the motion accuracy (for example, ±30 μm) of the robotic arm cannot meet the accuracy requirements (for example, ±10 μm) required by the system of the present application, the robotic arm is used to roughly find an implantation position, and the fine adjustment of the electrode position is still completed by two micro-operation motors.

In addition, the implantation apparatus 105 which is configured to implant a flexible electrode into a designated position of the operation area 106, includes an implantation needle, an implantation feeding mechanism and an implantation actuation mechanism. Wherein, the implanted needle mechanism is configured to engage a free end of an electrode with the needle tip portion so as to drive motion of the electrode. The implantation feeding mechanism is configured to move the implantation needle along a longitudinal direction of the implantation apparatus. The implantation actuation mechanism is configured to drive the implantation needle to insert the needle tip portion of the implantation needle into the operation area 106. Further, the implantation apparatus 105 may also be provided with an implantation motion mechanism for enabling the implantation apparatus 105 to implant the electrode from different angles and at different orientations.

FIG. 2 shows a non-limiting embodiment of a brain surface electrode implantation system. In this brain surface electrode implantation system 20, a binocular system is used, which mainly includes a first camera 201, a second camera 202, a vascular segmentation arithmetic unit 203, an implantation sequence determining unit 204, an implantation landing point prediction unit 205 and an implantation apparatus control unit 206. Wherein, the first camera 201 and the second camera 202 correspond to the cameras 101 and 102 in FIG. 1 respectively, and are configured to image the position and direction of the implantation apparatus relative to the operation area at an angle to each other, and similar features will not be described in detail here.

Specifically, the brain surface electrode implantation system 20 uses the first camera 201 and the second camera to image the brain surface and capture a first image 2010 and a second image 2020 respectively. The first image 2010 and the second image 2020 are imaging of the implantation apparatus and the operation area in different directions. As shown in FIG. 1, in a non-limiting example, if a three-dimensional coordinate system is established according to a control direction of the stepper motor in the motion control system, the position coordinates of the implantation apparatus and the operation area may be determined in the coordinate system based on the first image 2010 and the second image 2020 according to the first camera 201 and the second camera 202 at an angle to each other.

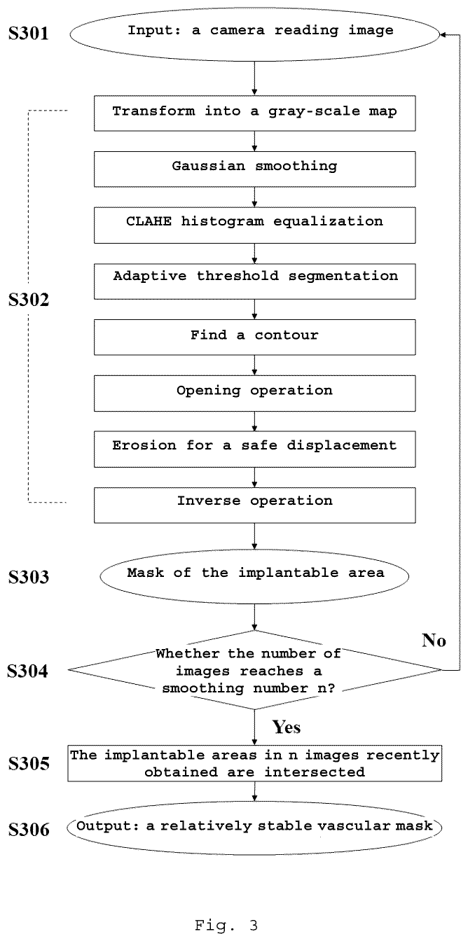

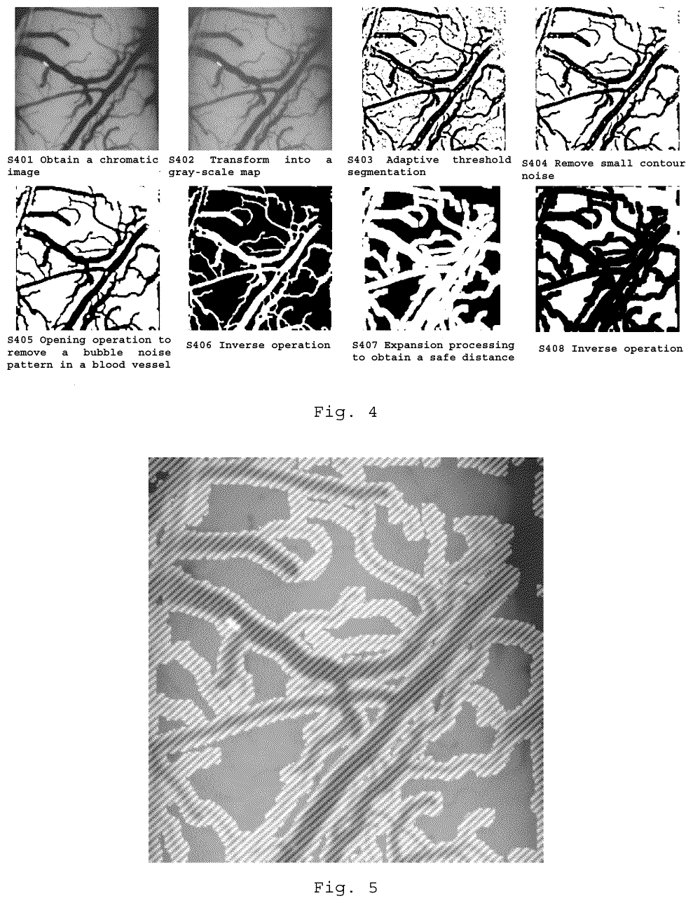

The vascular segmentation arithmetic unit 203 is configured to perform arithmetic processing on the first image 2010 and the second image 2020. The function mainly performed by the vascular segmentation arithmetic unit 203 is to obtain a vascular area mask of a brain surface based on the vascular segmentation algorithm, so as to determine the implantable area 2030 in the brain surface image. Generally, the vascular segmentation algorithm may be implemented in many methods, as described previously, including threshold partition, edge extraction and mathematical morphology processing. The algorithm used in the present application combines the advantages of several processing methods to eliminate the jitter of the video itself, process multi-frame images and obtain a smooth vascular image mask. FIG. 3 shows a non-limiting embodiment of the vascular segmentation algorithm, and FIG. 4 shows a schematic view of the results obtained by each step of processing of the vascular segmentation algorithm.

Specifically, in step S301 of FIG. 3, the first image 2010 and/or the second image 2020 are input into the vascular segmentation algorithm. Next, in step S302, a series of image processing steps are performed on the input image. First of all, the input image is transformed into a gray-scale map, and then segmentation processing is performed on an adaptive threshold. The contour of the blood vessel is found and the small contour noise is removed in a processed result. Next, the opening operation is performed to eliminate the bubble noise pattern belonging to the blood vessel in the original image, and subsequently inverse arithmetic processing is performed. In the processed result, expansion processing is performed on the blood vessel part to obtain a safe distance (also referred to as “corrosion” for a segment of safe displacement), and finally inverse arithmetic processing is performed again. At this time, the image mask of the implantable area is obtained in step S303. Further, judgment is set in step S304, so that a series of processing in S302 are repeated until the number of obtained images reaches a preset smoothing number n. After the above-described judgment, the implantable areas in n images recently obtained are intersected in step S305, and finally a relatively stable vascular image mask is output in step S306.

Correspondingly, FIG. 4 mainly shows an intermediate result obtained after each step of processing in a series of processing in step S302. As shown in the figure, in a result after transformation into a gray-scale map in S402, the interference of a vascular color is eliminated, and in a result after adaptive threshold segmentation in S403, the vascular and non-vascular areas are roughly divided. Because the adaptive threshold algorithm is used, it is not necessary to calculate a vascular gray-scale threshold in advance and prior data. In the case of appropriate imaging conditions, a stable result may be obtained. Next, after the contour noise is removed in S404 and the bubble noise pattern in the blood vessel is removed in S405, the change of the extracted contour edge is avoided as much as possible in the time domain, thereby improving the stability and safety of the contour extraction algorithm. Finally, after the processing of S406 to S408, the image area identified as a blood vessel has a reasonable safe distance, so as to minimize the risk of recognizing a blood vessel as an implantable area.

FIG. 5 shows an effect view of a vascular segmentation algorithm according to the above-described embodiments. As shown in the figure, the following image vascular analysis result is obtained in the 3 mm×3 mm macaque brain operation area. After inputting an original imaging result of the camera, an image recognition result is obtained after a series of algorithm processing, wherein the stripe shape mask is a segmented vascular area and the blank part is an implantable area. As may be seen from the figure, the vascular segmentation algorithm disclosed in the present application may effectively and stably recognize a vascular area during brain surface imaging, and ensure that the implantable area only contains a non-vascular portion with high accuracy.

In addition, the vascular segmentation algorithm disclosed in the present application may flexibly perform parameter adjustment. Generally, the algorithm parameters of the brain surface electrode implantation system may be adjusted based on a site of the implanted electrode. For example, when the requirements for the number of insertable points and the accuracy rate change, it is possible to affect an accuracy threshold of edge detection or a safe distance of expansion processing in the vascular segmentation algorithm. For the optical system, the change of the object distance between its lens and the operation area may result in that a specific area during imaging is enlarged or reduced, which further affects the number of sites to be detected, a site distance and an imaging resolution. Alternatively, the required number of electrode sites and the site distance may be designated by the user, automatically selected by the system or determined by the user assisted by the system, and the algorithm parameters may be adjusted on such basis.

Returning to FIG. 2, description will continue to be made. The implantation sequence determining unit 204 is configured to select at least one implantation position in the implantable area 2030. Particularly, in the case where a plurality of electrode implantation positions are required to be selected, the implantation sequence determining unit 204 calculates a distance between the implantation position and the electrode position according a known electrode position, so as to perform path planning on an electrode implantation sequence and obtain an electrode implantation sequence 2040. In a designated area determined based on the operation area, the motion control system controls a motion direction of the implantation apparatus through a stepper motor or a robotic arm by referring to an electrode direction, so that the electrodes are sequentially implanted in the determined positions. In order to prevent undesirable interaction between the electrodes, that is, the electrode that is being implanted may not exert an action force on the implanted electrode, it is necessary for the implantation sequence determining unit 204 to perform path planning according to the following principles: the electrode implanted later may not interfere with the electrode implanted earlier; the implanted electrode cannot be dragged during the movement process. Also that is, the desirable electrode implantation path should avoid instances such as crossing and transverse jumping as much as possible.

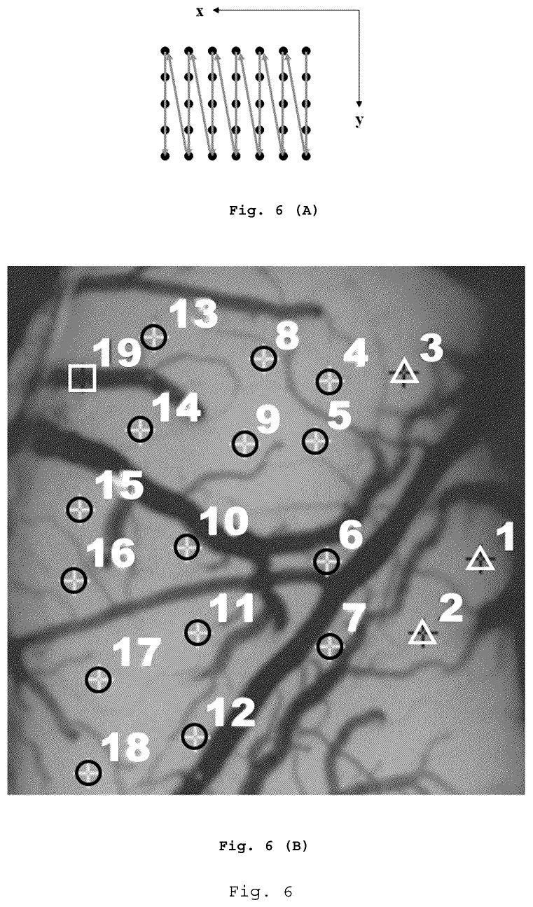

In one non-limiting embodiment, the sequence that may be used by the implantation sequence determining unit 204 is a sequence from near to far and from left to right of the implantation electrode with respect to the brain surface, as shown in FIG. 6. Taking the image processing result based on the vascular segmentation algorithm obtained in FIG. 5 as an example, FIG. 6 shows that an implantation position and an implantation sequence are determined according to the image processing result. FIG. 6(A) is a reference path planning sequence, wherein the implantable position of the brain surface and the distribution thereof obtained according to a series of processing described previously are simplified to a 5×7 lattice in a two-dimensional coordinate system, and the lattice of the implantation electrode (not shown) with respect to the brain surface is oriented from top to bottom (i.e., a positive direction of y axis in the figure), so that the sequence shown by the arrows among the lattices in 6(A) is obtained, that is, along a positive direction of x axis and along a positive direction of y axis. It is to be noted that, the number of lattices in FIG. 6(A) is only illustrative rather than restrictive. The example sequence of 6(A) is applied to the image processing result of FIG. 5, so as to obtain the path planning as shown in FIG. 6(B). FIG. 6(B) shows 19 calculated positions, wherein the position indicated by a square mark indicates an implantation site that is not recommended, the position indicated by a circular mark indicates an implantation site recommended by the algorithm, and the position indicated by a triangular mark indicates the implanted electrode site. The greatness of the number indicates the implantation sequence. As may be seen from the figure, the implantation sequences connected by these implantation positions are not crossed, and the states of the electrode sites are indicated by different marks to assist the observation and implantation process, so that the electrode implanted later may not interfere with and drag the electrode implanted earlier.

Next, the implantation landing point prediction unit 205 in FIG. 2 will continue to be described. The implantation landing point prediction unit 205 is configured to match the imaging of the first camera 201 and the second camera 202 to obtain a transformation matrix. Because the cameras may both obtain high-definition vascular imaging of the operation area, and the blood vessel has many features, the two cameras may be calibrated based on the feature matching of the data. The features such as SURF features or SIFT features may be used for matching in practical applications.

In the case of using a SURF feature, two cameras are matched based on the SURF feature to obtain an affine transformation matrix between the two cameras. Wherein, continuous Gaussian filters with different scales are used to process an image, and the feature points with a constant scale in the image are detected through a Gaussian difference. In addition, the Hessian matrix of spot detection is used to detect the feature points, and their determinant values represent the variations around the pixel points, so that the feature points are required to take the determinant values as a maximum value and a minimum value. In addition, in order to achieve a constant scale, SURF also uses the determinant value of the scale σ as detection of the feature points, and the Heisenberg matrix of a point p=(x,y) in a given graph at a scale σ is H (p,σ):

$$H ( p , σ ) = ( \begin{matrix}{L}_{xx} ( p , σ ) & {L}_{xy} ( p , σ ) \\ {L}_{xy} ( p , σ ) & {L}_{yy} ( p , σ )\end{matrix} )$$

Wherein, the function such Lₓₓ(p,σ) in the matrix is the gray-scale image after second-order differentiation. The 9×9 square filter which is taken as the lowest scale of SURF, approximates to a Gaussian filter with σ=1.2.

In the case of using a SIFT feature, with a high computational efficiency, it is possible to quickly perform image matching. Wherein, continuous Gaussian filters with different scales are used to process an image, and the feature points with a constant scale in the image are detected through a Gaussian difference. SURF uses a square filter instead of a Gaussian filter in SIFT, so as to achieve the approximation of Gaussian blur. Its filter may be expressed as:

$$S ( x , y ) = {\sum }_{i = 0}^{x} {\sum }_{j = 0}^{y} I ( i , j )$$

In addition, the use of the square filter may greatly improve the operation speed by using an integral image, and it is only necessary to calculate four corner values of the square filter.

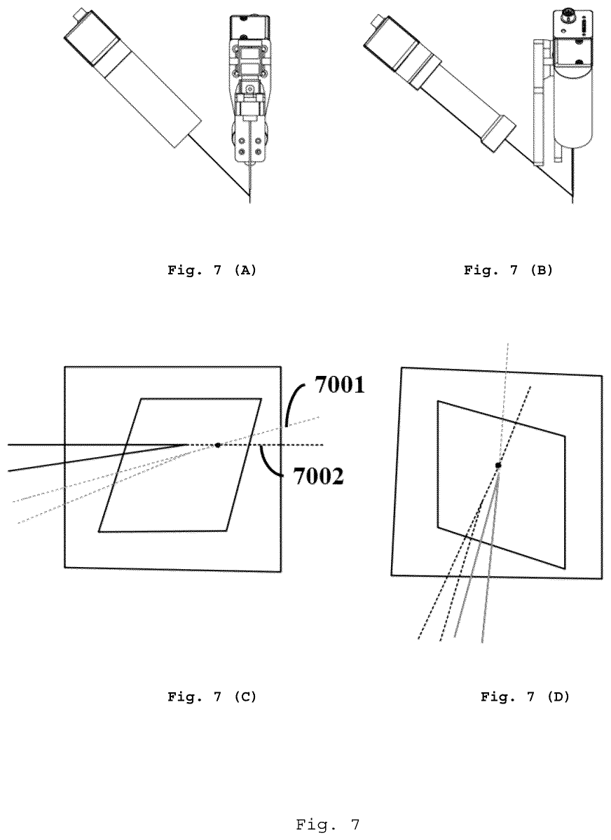

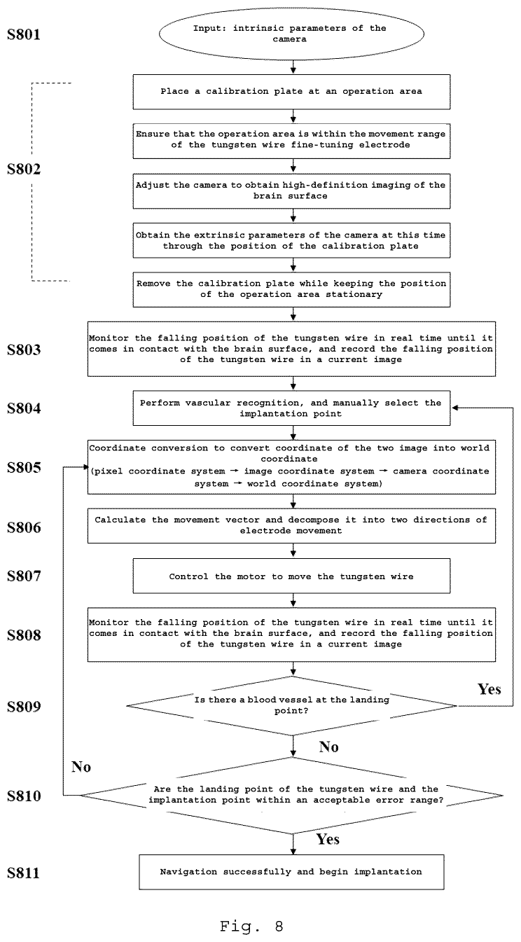

After feature matching, the first camera 201 and the second camera 202 are calibrated, that is, for any group or groups of first images 2010 and second images 2020, their correspondence and transformation relationships are obtained. On such basis, FIG. 7 further shows a schematic view of landing point prediction of the implantation tool.

In one non-limiting embodiment, when the implantation apparatus appears in the imaging of the binocular system, the two cameras may each position a straight line where the implantation tool is situated during their imaging. Also that is, the first camera 201 and the second camera 202 may determine a plane where the implantation tool is situated in the three-dimensional space (and/or the established spatial coordinate system) respectively. Because of different viewing angles of the two cameras, the image and the position information obtained during their imaging are not exactly the same. Taking the first camera 201 capturing the first image 2010 and the second camera 202 capturing the second image 2020 as an example, FIG. 7 (A) and FIG. 7 (B) show schematic views of projecting any camera of the first camera 201 and the second camera 202 into the imaging direction of the other camera respectively. FIG. 7 (C) shows a schematic view of the implantation position in the second image 2020. The implantation landing point prediction unit 205 projects the first straight line 7001 where the implantation position is situated in the first image 2010 into the second image 2020. If the implantation position itself falls in the second straight line 7002 in the second image 2020, the intersection point between the first straight line 7001 and the second straight line 7002 will be determined as a predicted landing point 2050 of the implantation apparatus in the second image 2020. Similarly, FIG. 7 (D) shows a predicted landing point of the implantation position in the first image 2010. Because of the calculation error of the transformation relationship between the two cameras, the two intersection points obtained might not be completely coincident in an actual image, but it may be determined that an actual predicted landing point is in the vicinity of these two intersection points.

Continuing to return to FIG. 2, the implantation apparatus control unit 206 will be described. Based on the implantation sequence 2040 determined by the implantation sequence determining unit 204 and the predicted landing point 2050 determined by the implantation landing point prediction unit 205, the implantation apparatus control unit 206 finally controls the implantation apparatus in real time according to the predicted landing point 2050 sequentially based on the implantation sequence 2040 until the implantation point coincides with the predicted landing point. The implantation apparatus control unit 206 may use full-supervision or semi-supervision control, and make different selections as chosen by the user, so as to achieve a personalized control effect.

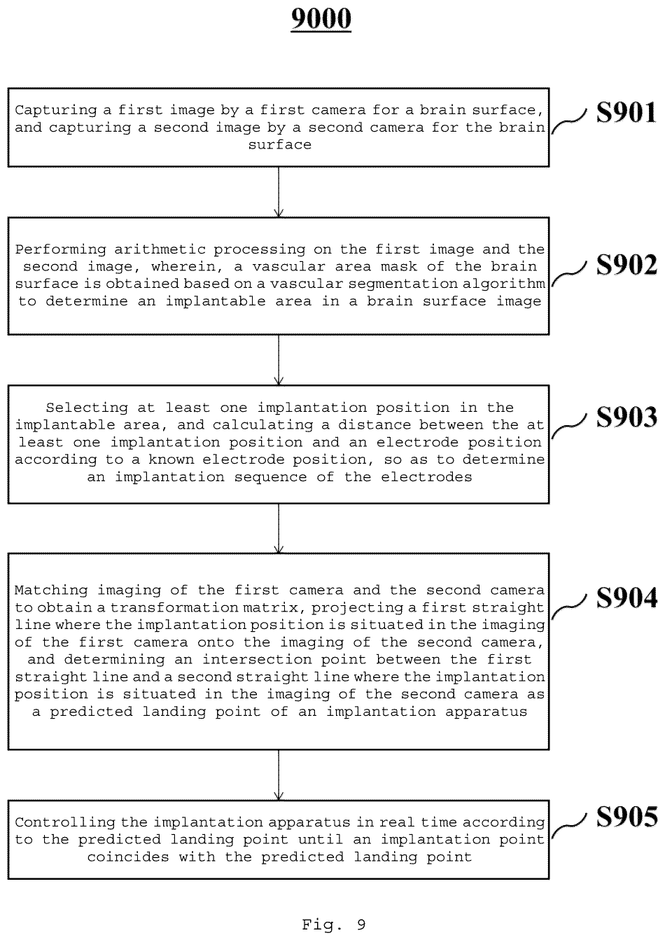

FIG. 8 shows a flowchart of an implantation tool control algorithm in one non-limiting embodiment. The control algorithm takes the intrinsic parameters of the camera in the optical system as an input in step S801. First of all, a series of processing in step S802 are performed. The calibration plate is placed in the operation area and it is ensured that the operation area is within the movement range of the tungsten wire fine-tuning electrode. At this time, the imaging condition of the camera is adjusted to render high-definition imaging of the brain surface. After the extrinsic parameters of the camera are obtained through the position of the calibration plate, the calibration plate is removed, and the position of the operation area is kept stationary. Next, in step S803, the falling position of the tungsten wire is monitored in real time until it comes in contact with the brain surface, and the falling position of the tungsten wire in a current image is recorded. In step S804, vascular recognition is performed, and the implantation point is selected manually based on the restriction of the implantation sequence 2040, or selected automatically or assisted in calculation by the system. Next, coordinate conversion is performed in step S805 to convert the two image coordinates into world coordinates. Specifically, conversion is performed according to “pixel coordinate system→image coordinate system→camera coordinate system→world coordinate system”. In step S806, the movement vector of the implantation apparatus relative to the electrode is calculated and decomposed into two directions of electrode movement, for example, finely controlling the movements of two micro-stepper motors in the x and y directions beyond the z axis direction of the stepper motor. Subsequently, in step S807, the motors in different directions described previously are controlled to move the tungsten wire to be implanted. Step S808 repeats the processing of step S803.

Next, one judgment is performed at step S809, and when there is no blood vessel at the landing point of the tungsten wire (it is judged to be “No” at step S809), continue to process with another judgment at step S810, that is, whether the difference between the landing point of the tungsten wire and the implantation point is within an acceptable error range. If it is judged to be “Yes” in step S809, that is, there is a blood vessel at the landing point of the tungsten wire in a current image, return to S804 to select a new implantation point that meets the requirements. Subsequently, at step S810, if the judgment result is Yes, it indicates that the control algorithm of the implantation tool navigates successfully, so that electrode implantation may begin. If the judgment result in step S810 is No, it means that the error between the landing position of the tungsten wire in a current image and the selected implantation point is unacceptable, return to S805 and perform coordinate conversion again to obtain a more accurate landing position.

FIG. 9 is a schematic view showing steps of a machine vision based electrode implantation method according to embodiments of the present disclosure. Based on the content described previously, the electrode implantation method 9000 mainly includes the following steps: at step S901, a first image is captured by a first camera for the brain surface, and a second image is captured by a second camera for the brain surface. At step S902, the first image and the second image are processed by operation, wherein the vascular area mask of a brain surface is obtained based on the vascular segmentation algorithm to determine the implantable area in the brain surface image. Next, at step S903, at least one implantation position is selected in the implantable area, and the distance between the at least one implantation position and an electrode position is calculated according to a known electrode position, so as to determine an implantation sequence of the electrodes. Subsequently, in step S904, the imaging of the first camera and the second camera are matched to obtain a transformation matrix, the first straight line where the implantation position is situated in the imaging of the first camera is projected into the imaging of the second camera, and the intersection point between the first straight line and the second straight line where the implantation position is situated in the imaging of the second camera is determined as a predicted landing point of the implantation apparatus. Finally, in step S905, the implantation apparatus is controlled in real time according to the predicted landing point until the implantation point coincides with the predicted landing point.

Alternatively, the machine vision based electrode implantation method and system disclosed in the present application may also have other implementations.

In one example, the brain surface electrode implantation system disclosed in the present application does not necessarily need to use a binocular system with two cameras for calibration, and a monocular system may also control a position in the implantation tool plane. The following two implementations may be used, that is, vertical observation and oblique observation. In the case of vertical observation, the camera shoots vertically above the brain surface to obtain a stationary image. In this image, vascular segmentation is performed, and the position of the implantation apparatus is controlled by the transformation of pixel coordinates and manipulator coordinates, and the implantation apparatus is moved above the brain surface to perform implantation. In this implementation, an observation camera obliquely placed may be added to facilitate the observation of an implantation condition. In the case of oblique observation, the camera is placed obliquely relative to the brain surface to obtain a screen with an inclination angle, and the position of the implantation apparatus is obtained through coordinate transformation, and the electrode is controlled to move its position for implantation. In contrast, due to the problem of an imaging angle, the vertical observation method has better control accuracy and vascular segmentation accuracy than the oblique observation method.

If a monocular system is used, a calibration plate method and a nine-point calibration method are mainly used to realize the target requirements of this system during the calibration. The calibration plate method means placing a calibration plate at a position close to the operation area to obtain the extrinsic parameters of the camera, and then calculating a conversion relationship between the image and the actual coordinates. The nine-point calibration method means capturing a brain area at a certain position, and then controlling the tungsten wire to move to the designated nine points, and recording the position of the tungsten wire in the image and the position in the actual coordinate system at this time respectively, so as to obtain a coordinate transformation relationship. Based on the conversion relationship, the displacement that the tungsten wire should move at an actual position may be calculated by the distance between the landing point of the tungsten wire and the target point in the image. Compared with the binocular system, these two methods of the monocular system both require a stationary height position of the brain surface from the camera, which may affect its accuracy. In practical application, because the brain surface itself is not a plane, it is necessary for the camera to adjust a height in real time, that is, to reach the same position above the brain surface with micron-level precision.

The words “front”, “rear”, “top”, “bottom”, “above” and “below” in the specification and claims, if any, are used for descriptive purposes but not necessarily for describing a constant relative position. It should be understood that, the words thus used are interchangeable where appropriate, so that the embodiments of the present disclosure described here can, for example, perform operations in other orientations than those shown or otherwise described here.

As used here, the word “exemplary” means “serving as an example, instance or illustration”, rather than as a “model” to be accurately reproduced. Any implementation exemplarily described here is not necessarily to be construed as preferable or advantageous over other implementations. Moreover, the present disclosure is not defined by any expressed or implied theory provided in the above-described technical field, background, summary or detailed description.

As used here, the word “substantially” means including any slight change caused by design or manufacturing defects, tolerances of devices or elements, environmental influences and/or other factors. The word “substantially” also allows for differences from perfect or ideal instances caused by parasitic effects, noise and other practical considerations that might be present in actual implementations.

For reference purposes only, similar terms such as “first” and “second” may be used herein, and thus are not intended to be restrictive. For example, the words “first”, “second” and other such numerical words involving the structures or elements do not imply an order or sequence unless specified otherwise in the context.

It should also be understood that, the words “comprising/including” when used herein indicate the presence of the features, wholes, steps, operations, units and/or components as set forth, but do not exclude the presence or addition of one or more other features, wholes, steps, operations, units and/or components and/or combinations thereof.

As used herein, the terms “and/or” include any and all combinations of one or more of the associated listed items. The terms used herein which are for the purpose of describing specific embodiments only, are not intended to limit the present disclosure. As used herein, the singular forms “a”, “an” and “the” are also intended to include plural forms unless otherwise clearly indicated in the context.

Those skilled in the art should be appreciated that, the boundaries between the above-described operations are merely illustrative. Multiple operations may be combined into a single operation, which may be distributed among additional operations, and performed at least partially overlapping in time. Moreover, alternative embodiments may include multiple examples of specific operations, and the operation sequence may be changed in other various embodiments. However, other modifications, changes and substitutions are also possible. Therefore, this specification and the accompanying drawings should be regarded as illustrative rather than restrictive.

Although some specific embodiments of the present disclosure have been described in detail by way of examples, those skilled in the art should understand that the above examples are only for an illustrative purpose, rather than limiting the scope of the present disclosure. Various embodiments disclosed here may be arbitrarily combined without departing from the spirit and scope of the present disclosure. Those skilled in the art should also understand that, multiple modifications may be made to the embodiments without departing from the scope and spirit of the present disclosure. The scope of the present disclosure is defined by the appended claims.

What is claimed is:

1. A machine vision based electrode implantation method, comprising:
capturing a first image by a first camera for a brain surface, and capturing a second image by a second camera for the brain surface;
performing arithmetic processing on the first image and the second image, wherein, a vascular area mask of the brain surface is obtained based on a vascular segmentation algorithm to determine an implantable area in a brain surface image;
selecting at least one implantation position in the implantable area, and calculating a distance between the at least one implantation position and an electrode position according to a known electrode position, so as to determine an implantation sequence of the electrodes;
matching imaging of the first camera and the second camera to obtain a transformation matrix, projecting a first straight line where the implantation position is situated in the imaging of the first camera onto the imaging of the second camera, and determining an intersection point between the first straight line and a second straight line where the implantation position is situated in the imaging of the second camera as a predicted landing point of an implantation apparatus; and
controlling the implantation apparatus in real time according to the predicted landing point until an implantation point coincides with the predicted landing point.

2. The electrode implantation method according to claim 1, wherein:
full-supervision or semi-supervision control is performed on the implantation apparatus according to the predicted landing point.

3. The electrode implantation method according to claim 1, wherein:
the first camera and the second camera have the same imaging plane.

4. The electrode implantation method according to claim 3, wherein:
the first camera and the second camera form an included angle of about 90° on a horizontal projection.

5. The electrode implantation method according to claim 1, wherein:
each of the first camera and the second camera comprises an optical system respectively, and the first camera and the second camera are coupled to a motion control system respectively.

6. The electrode implantation method according to claim 5, wherein:
the optical system comprises an external light source which uniformly illuminates the brain surface.

7. The electrode implantation method according to claim 6, wherein:
the external light source has a wavelength range of 495 nm to 570 nm.

8. The electrode implantation method according to claim 5, further comprising:
performing image processing by the first camera and the second camera respectively, and merging the image-processed data from the first camera and the second camera; and
determining the coordinates of the implantation apparatus in the imaging of the first camera and the second camera according to the merged data.

9. The electrode implantation method according to claim 8, wherein:
the motion control system controls movement of the implantation apparatus according to the coordinates.

10. The electrode implantation method according to claim 5, wherein:
the motion control system comprises three stepper motors configured to control the implantation apparatus to move in an area substantially parallel to the electrode implantation area.

11. The electrode implantation method according to claim 1, wherein the cerebrovascular segmentation algorithm comprises following steps:
transforming the brain surface image into a gray-scale map;
segmenting the gray-scale map according to an adaptive threshold;
removing small contour noise from the segmented gray-scale map;
performing opening operation to remove a bubble noise pattern in a blood vessel;
performing inverse operation;
performing expansion processing to obtain a safe distance at a boundary of a vascular area; and
performing inverse operation again.

12. The electrode implantation method according to claim 11, wherein:
parameters in the cerebrovascular segmentation algorithm are adjusted based on a number of sites to be detected, a site distance and an imaging resolution.

13. The electrode implantation method according to claim 1, wherein:
the implantation sequence of the electrodes is determined so that an electrode being implanted does not apply an action force on an implanted electrode.

14. The electrode implantation method according to claim 13, wherein:
paths for implanting the electrodes are planned based on the implantation sequence of the electrodes, wherein the paths are not crossed.

15. The electrode implantation method according to claim 1, wherein:
feature matching of data is performed on the first camera and the second camera for calibration.

16. The electrode implantation method according to claim 15, wherein:
a square filter is used to realize an image processing effect of Gaussian blur.

17. The electrode implantation method according to claim 1, wherein:
the implantation apparatus comprises an implantation needle, an implantation feeding mechanism and an implantation actuation mechanism,
wherein the implantation needle is configured to engage a free end of an electrode with a needle tip portion thereof so as to drive motion of the electrode,
the implantation feeding mechanism is configured to move the implantation needle along a longitudinal direction of the implantation apparatus, and
the implantation actuation mechanism is configured to drive the implantation needle to insert the needle tip portion of the implantation needle into the brain.

18. The electrode implantation method according to claim 17, wherein:
the implantation apparatus is provided with an implantation motion mechanism configured to enable the implantation apparatus to implant the electrode from different angles and at different orientations.

19. A machine vision based electrode implantation system, comprising:
a first camera configured to capture a first image for a brain surface;
a second camera configured to capture a second image for a brain surface;
a vascular segmentation arithmetic unit configured to perform arithmetic processing on the first image and the second image, wherein a vascular area mask of the brain surface is obtained based on a vascular segmentation algorithm to determine an implantable area in a brain surface image;
an implantation sequence determining unit configured to select at least one implantation position in the implantable area and calculate a distance between the at least one implantation position and an electrode position according to a known electrode position, so as to determine an implantation sequence of the electrodes;
an implantation landing point prediction unit configured to match imaging of the first camera and the second camera to obtain a transformation matrix, project a first straight line where the implantation position is situated in the imaging of the first camera onto the imaging of the second camera, and determine an intersection point between the first straight line and a second straight line where the implantation position is situated in the imaging of the second camera as a predicted landing point of an implantation apparatus; and
an implantation apparatus control unit configured to control the implantation apparatus in real time according to the predicted landing point until an implantation point coincides with the predicted landing point.

20. The electrode implantation system according to claim 19, wherein:
full-supervision or semi-supervision control is performed on the implantation apparatus according to the predicted landing point.

21. The electrode implantation system according to claim 19, wherein:
the first camera and the second camera have the same imaging plane.

22. The electrode implantation system according to claim 19, wherein:
each of the first camera and the second camera comprises an optical system respectively, and the first camera and the second camera are coupled to a motion control system respectively.

23. The electrode implantation system according to claim 22, wherein:
the optical system comprises an external light source which uniformly illuminates the brain surface.

24. The electrode implantation system according to claim 19, wherein:
performing image processing by the first camera and the second camera respectively, and merging the image-processed data from the first camera and the second camera; and
determining the coordinates of the implantation apparatus in the imaging of the first camera and the second camera according to the merged data.

25. The electrode implantation system according to claim 24, wherein:
the motion control system controls movement of the implantation apparatus according to the coordinates.

26. The electrode implantation system according to claim 19, wherein the cerebrovascular segmentation algorithm comprises the following steps:
transforming the brain surface image into a gray-scale map;
segmenting the gray-scale map according to an adaptive threshold;
removing small contour noise from the segmented gray-scale map;
performing opening operation to remove a bubble noise pattern in a blood vessel;
performing inverse operation;
performing expansion processing to obtain a safe distance at a boundary of a vascular area; and
performing inverse operation again.

27. The electrode implantation system according to claim 26, wherein:
the implantation sequence determining unit is further configured to determine the implantation sequence of the electrodes so that an electrode being implanted does not apply an action force on an implanted electrode.

28. The electrode implantation system according to claim 27, wherein:
paths for implanting the electrodes are planned based on the implantation sequence of the electrodes, wherein the paths are not crossed.

29. The electrode implantation system according to claim 19, wherein:
feature matching of data is performed on the first camera and the second camera for calibration.

30. The electrode implantation system according to claim 19, wherein:
the implantation apparatus comprises an implantation needle, an implantation feeding mechanism and an implantation actuation mechanism,
wherein the implantation needle is configured to engage a free end of an electrode with a needle tip portion thereof so as to drive motion of the electrode,
the implantation feeding mechanism is configured to move the implantation needle along a longitudinal direction of the implantation apparatus, and
the implantation actuation mechanism is configured to drive the implantation needle to insert the needle tip portion of the implantation needle into the brain.
